(19) United States
(12) Patent Application Publication
(10) Pub. No.: US 2026/0158287 A1
(43) Pub. Date: Jun. 11, 2026

(54) HEALING AND REJUVENATION SYSTEM

(71) Applicant: Morgan William Rogers, Corona del Mar, CA (US)

(72) Inventor: Morgan William Rogers, Corona del Mar, CA (US)

(21) Appl. No.: 19/281,608

(22) Filed: Jul. 26, 2025

Related U.S. Application Data
(60) Provisional application No. 63/676,312, filed on Jul. 26, 2024.

(51) Int. Cl. A61N 2/00; A61H 9/00; A61H 23/00; A61H 23/02

(52) U.S. Cl. CPC A61N 2/002; A61H 9/0092; A61H 23/006; A61H 23/0218; A61N 2/006; A61H 2201/107; A61H 2201/5005; A61H 2209/00

(57) ABSTRACT

A healing and rejuvenation system is provided for treating a user of the system. The system has a percussive-impacting mechanism that generates an acceleration on the user, an inspired-gas delivery unit that controls the partial pressure of a gas inhaled by the user, a vascular-constriction device that compresses a blood vessel of the user, and a nerve-plexus-stimulation device that generates a magnetic field adjacent a nerve plexus of the user.

### CROSS-REFERENCE TO RELATED APPLICATION

This application claims priority to U.S. Provisional Application No. 63/676,312, filed Jul. 26, 2024, and entitled “HEALING AND REJUVENATION SYSTEM,” the disclosure of which is hereby incorporated by reference in its entirety.

### BACKGROUND

Current aesthetic procedures often debride the skin and then employ healing regimens that are directed to topically treating the damaged skin. However, the body possesses a powerful healing and rejuvenation ability that, if properly harnessed, can produce far superior results compared to healing regimens that are directed to only one or two isolated abilities of the body to heal itself. The present disclosure is directed to a sophisticated healing and rejuvenation system that utilizes a multi-pronged and synergistic approach to treat human patients. The methods and systems disclosed herein are useful not only for healing skin after an aesthetic procedure, but also can be used to improve cardiovascular health, reverse age-related tissue damage, and improve cancer treatment outcomes.

### SUMMARY

Disclosed herein are embodiments of a rejuvenation and healing system that employs contemporaneous stimulation of two or more reactive oxygen species (ROS)-producing pathways of the user of the system. In some arrangements, the system can employ a vibration-generating mechanism (also referred to herein as a percussive-impacting mechanism) to impose a vibration onto the body of the user of the system. In some arrangements, the system can include an inspired-gas delivery unit that controls the partial pressure of one or more gases inhaled by the user of the system. In some arrangements, the system can include a vascular-constriction device (e.g., an oscillometric device) configured to compress a blood vessel of a user of the system. In some arrangements, the system can include a nerve-plexus-stimulation device configured to emit a strong magnetic field adjacent a nerve plexus of a user of the system. Also disclosed herein are methods of treating a person with the healing and rejuvenation system by contemporaneously stimulating with the system two or more ROS-producing physiological pathways of the user of the system.

### BRIEF DESCRIPTION OF THE DRAWINGS

Embodiments of the present disclosure will now be described hereinafter, by way of example only, with reference to the accompanying drawings in which:

FIG. 1 depicts an illustrative, non-limiting example of a healing and rejuvenation system, according to some aspects of the present disclosure.

FIG. 2 depicts an illustrative, non-limiting example of a healing and rejuvenation system, according to some aspects of the present disclosure.

FIG. 3A depicts an illustrative, non-limiting example of a vascular compression device, according to some aspects of the present disclosure.

FIG. 3B depicts schematically an artery compressed by a vascular compression device of FIG. 3A, according to some aspects of the present disclosure.

### DETAILED DESCRIPTION

### Overview

This disclosure relates generally to a healing and rejuvenation system that stimulates simultaneously different healing pathways of the body. Without being bound to theory, a possible explanation for the superior therapeutic results achieved with the system of the present disclosure may arise from the simultaneous stimulation of different physiologic pathways for moderating the production of reactive oxygen species (ROS). ROS not only damage tissue due to the reactivity of the ROS, but also ROS are unstable and have extremely short half-lives. Therefore, the simultaneous stimulation of different physiologic pathways by the present system, as disclosed herein, may achieve superior results by enabling different cellular and molecular pathways of the human body to operate together in real-time to better modulate short-lived and tissue-damaging ROS compared with other methods of treatment that activate, one at a time, various physiologic healing pathways.

In some aspects, the system disclosed herein can improve oxygen delivery and oxygen off-loading in a target tissue of the body, as described herein. In some aspects, the healing and rejuvenation system can accelerate healing times or stimulate skin rejuvenation.

### Whole-Body Therapeutic Systems

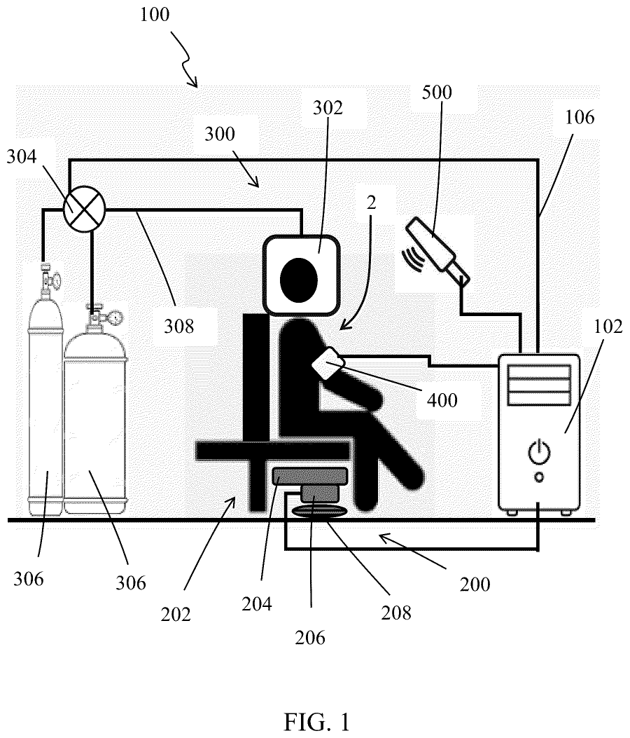

FIG. 1 depicts a non-limiting, illustrative arrangement of a healing and rejuvenation system 100, according to some aspects of the present disclosure. As shown in FIG. 1, the system 100 can be used to treat a patient 2. The system 100 can include a system-control unit 102 that is configured to control different components of the healing and rejuvenation system 100, as described herein. The system 100 can include a percussive-impacting mechanism 200 configured to impart a mechanical loading regimen on the patient 2 by way of the percussions or vibrations generated by the percussive-impacting mechanism 200. In some embodiments, the percussive impact stimulation can be achieved instead by a vibration-generating device (e.g. an off-center flywheel). Percussive and vibrational treatment has been found in microgravity environments to improve bone density. Thus, one possible explanation for the beneficial effects observed with the percussive-impacting mechanism 200 of the healing and rejuvenation system 100 of the present disclosure may be that the vibrations stimulate mechano-loading responses in bone cells (e.g., osteoblasts, osteoclasts). The percussive-impacting mechanism 200 may also enhance oxygen delivery and oxygen off-loading by enhancing fluid diffusion in the microenvironments of the body (e.g., bone canaliculi, capillary beds of skin and other tissues). In some arrangements, the percussive-impacting mechanism 200 can be configured to deliver to a user the system 100 a vibration having a frequency within the range between 10 Hz and 200 Hz. In some arrangements, the percussive-impacting mechanism 200 can be configured to deliver to a user the system 100 a vibration having an amplitude or magnitude sufficient to impose on the user of the system 100 a force between the range of 0.1 g and 10 g, where g denotes the Earth's gravitational force (i.e., 9.8 m/s²).

In the system 100 of FIG. 1, the percussive-impacting mechanism 200 is configured as a chair 202 on which the patient 2 can sit to receive treatment from the system 100. The percussive-impacting mechanism 200 can be configured as a magnetic drive unit 204 that is controlled (e.g., by the control unit 102) to pull a striker rod 206 away from a restoring spring 208 and through the core of the magnetic drive unit 204 to impact the striker rod 206 directly or indirectly upon the chair 202, thereby imparting on the patient 2 a mechanical loading regimen that is under precise control (e.g., by the control unit 102). As can be appreciated with reference to FIG. 1, in some arrangements the percussive-impacting mechanism 200 can be configured as a platform on which the patient 2 stands to receive treatment from the percussive-impacting mechanism 200. In some arrangements, the percussive-impacting mechanism 200 can be configured as a bed on which the patient 2 lies down to receive treatment. The control unit 102 can be configured to control the frequency and amplitude of the percussive impacts or vibrations generated by the percussive-impacting mechanism 200. In the illustrated system 100 of FIG. 1, the control unit 102 is shown hard-wired to different components of the system 100 through a wire connection 106. However, in some arrangements, the control unit 102 can control a component of the system 100 through a wireless connection (e.g., Bluetooth, WiFi).

With continued reference to FIG. 1, the system 100 can further include an inspired-gas delivery unit 300 configured to control the pressure and mixture of gases that are inhaled by the patient 2 during treatment with the system 100, as described herein. In the system 100 of FIG. 1, the inspired-gas delivery unit 300 is configured as a hood 302 that covers the head of the patient 2. In some arrangements, the inspired-gas-delivery unit 300 can deliver gas to the patient 2 through a nose cannula or face mask that does not cover the head of the patient 2. The inspired-gas delivery unit 300 can include a regulator 304 that regulates the mixture of one or more supply gases 306 that are delivered to the hood 302 through a supply line 308. The control unit 102 can be configured to control the regulator 304 to control the partial pressure of the gases inspired by the patient 2. In some arrangements, the hood 302 can be used to introduce the inspired gases to the patient 2 at a pressure different than atmospheric pressure. In some arrangements, the system 100 is configured to deliver pressurized, highly-oxygenated gas that is balanced with nitrogen. In some aspects, providing the patient 2 with highly-oxygenated gas under high pressure can improve oxygen delivery into the tissue targeted for therapy with the system 100. In some arrangements, the gas regulated by the inspired-gas delivery unit 300 is selected from the group consisting of: oxygen, air, nitrogen, and carbon dioxide.

In some aspects, the system 100 can include a vascular-constriction device 400, as shown in FIG. 1. The vascular-constriction device 400 is shown as a cuff that wraps around the upper arm of the patient 2 in FIG. 1. However, in some arrangements, the vascular-constriction device 400 can be applied to a different body part of the patient 2 (e.g., forearm, shin, thigh). As described herein, the vascular-constriction device 400 can be configured to compress the tissue of the patient 2 with sufficient force to occlude the low-pressure venous return blood flow while allowing the higher-pressure arterial blood flow to occur. The vascular-constriction device 400 can improve oxygen offloading by delivering highly-oxygenated blood into tissues targeted for therapy with the system 100. In some arrangements, the vascular-constriction device 400 can provide a tissue compression force within the range between 50 mmHg and 250 mmHg. In some arrangements, the vascular-constriction device 400 can vary the tissue compression force over time (e.g., following a sinusoidal waveform).

In some aspects, the system 100 can include a nerve-plexus-stimulation device 500, as shown in FIG. 1. The nerve-plexus-stimulation device 500 can include a magnetic-field generator that generates a strong magnetic field. In some arrangements, the nerve-plexus-stimulation device 500 can emit a magnetic field having a strength between 5 mT and 1.0 T. In some arrangements, the nerve-plexus-stimulation device can also transmit vibrational forces to the patient 2 when the nerve-plexus-stimulation device 500 is brought into contact the patient 2. It has been found that the heart rate of the patient 2 can be affected by placing the nerve-plexus-stimulation device 500 near a nerve center (e.g., solar plexus) of the patient 2.

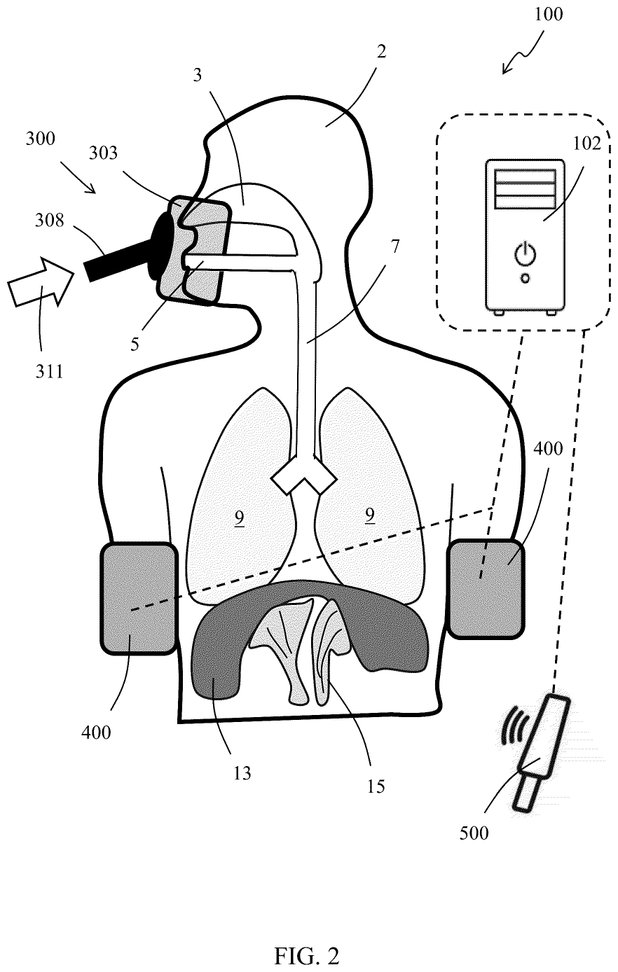

FIG. 2 depicts a non-limiting, illustrative arrangement of a healing and rejuvenation system 100, according to some aspects of the present disclosure. As illustrated in FIG. 2, the inspired-gas delivery unit 300 can be configured as a breathing mask 303 configured to cover a nasal airflow passage 3 and a mouth airflow passage 5 of the patient 2. The nasal airflow passage 3 and mouth airflow passage 5 converge into a brachial trunk 7 that later bifurcates to connect to the lungs 9 of the patient 2. The inspired-gas delivery unit 300 can allow precise control of the pressure and composition of inspiration gas 311 delivered to the mask 303 through the supply line 308, as described herein. The inspiration gas 311 is inhaled by the patient 2 at the mask 303, fills the nasal airflow passage 3 and the mouth airflow passage 5, and is conveyed to the lungs 9 through the brachial trunk 7. As can be appreciated from FIG. 2, the mask 303 can be sized, in some arrangements of the system 100, to cover only one of the nasal airflow passage 3 or the mouth airflow passage 5 while leaving the other uncovered.

FIG. 2 further illustrates that the system 100 can include a control unit 102 configured to communicate wirelessly with the vascular-constriction device 400 and the nerve-plexus-stimulation device 500 (as indicated by dashed lines in FIG. 2). In some aspects, the nerve-plexus-stimulation device 500 can be configured to deliver mechanical vibrational forces and strong magnetic field forces to a muscle 13 or to a nerve plexus 15 of the patient 2.

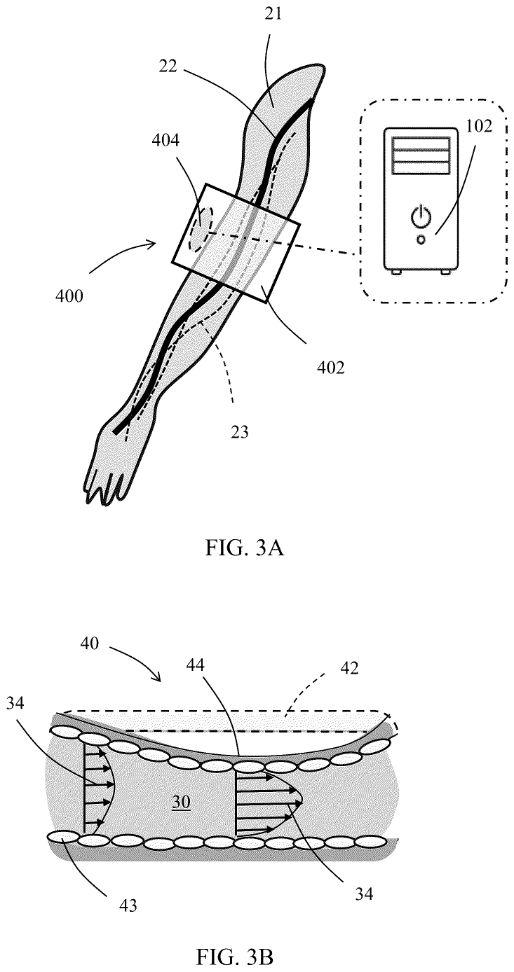

FIG. 3A depicts a non-limiting, illustrative arrangement of a vascular-constriction device 400 of a healing and rejuvenation system 100, according to some aspects of the present disclosure. The vascular-constriction device 400 of FIG. 3A is depicted as a cuff 402 that circumferentially surrounds the bicep area of an arm 21 of the patient. The vascular-constriction device 400 can include an adjustable bladder 404. The adjustable bladder 404 can be under the control of the control unit 102, as illustrated in FIG. 3A by the dash-dot lines indicating a wireless connection between the control unit 102 and the adjustable bladder 404. The pressure in the adjustable bladder can be increased by a pump (not shown) that can be mounted on or within the cuff 402. Inflation of adjustable bladder 404 can increase the pressure on the vasculature of the arm 21. In some arrangements, the control unit 102 can monitor the pressure within the adjustable bladder 404 and adjust the pump of the cuff 402 accordingly to achieve control of the pressure exerted on the vasculature of the arm 21. In some aspects, the pressure exerted on the vasculature of the arm 21 can be balanced to allow an artery 22 to remain open (as indicated by the heavy line in FIG. 3A) while a vein 23 of the arm 21 is closed (as indicated by dashed lines in FIG. 3A). In this way, super-oxygenated blood (e.g., resulting from inspiration of high-oxygen gas) can be driven into tissue. In some arrangements, the vascular-constriction unit 400 can include a pulse oximeter (not shown) to measure blood oxygen saturation. In some arrangements, the vascular-constriction unit 400 can be configured to operate as a volume-clamp plethysmograph.

FIG. 3B depicts an axial-cross-sectional view of an artery 40 undergoing compression from a vascular-constriction device 400, according to some aspects of the present disclosure. Under pressure (e.g., from inflation of the adjustable bladder 404), the artery 40 collapses from an uncompressed shape 42 to a compressed shape 44. As depicted in FIG. 3B, the blood 30 flowing through the artery 40 accelerates as the blood 30 passes through the compressed shape 44 (as indicated by a lengthening of the axial velocity vectors 34 in FIG. 3B). The artery 40 is lined with endothelial cells 43 that are in direct contact with the flowing blood 30 and are sensitive to fluid shear stress. Endothelial cells 43 also possess the ability to synthesize nitric oxide (NO) through an endothelial-cell nitric-oxide-synthase (eNOS) expressed on the cell membrane of endothelial cells 43. Further, endothelial cells 43 participate in regulation of vascular tone by producing NO to vasodilate the smooth muscle cells (not shown) of the arterial wall in response to the fluid shear forces detected by the endothelial cell 43. The fluid shear stress at the arterial wall is the product of the blood viscosity (μ) and the derivative of the axial-velocity profile (du/dx) at the arterial wall. As can be appreciated from FIG. 3B, the vascular-constriction device 400 of the present system 100 can be configured to elicit the endothelial cells 43 to release NO. Further, this NO is released into a jet-like accelerated flow that has enhanced dispersion dynamics, likely enhancing downstream delivery of the endothelial-cell-produced NO. In the present system 100, this NO is released real-time while the system 100 is simultaneously activating other healing and rejuvenation pathways of the body. In this way, the present system 100 enables a multi-pronged and sophisticated approach to harness the body's own healing pathways, which achieves better therapeutic outcomes compared to other approaches that activate healing pathways on a one-by-one basis.

### Other Variations and Terminology

While certain embodiments have been described, these embodiments have been presented by way of example only and are not intended to limit the scope of protection. Indeed, the novel methods and systems described herein may be embodied in a variety of other forms. It will be further understood by those skilled in the art that the present disclosure extends beyond the specifically disclosed embodiments to other alternative embodiments or uses and obvious modifications and equivalents thereof, including embodiments which do not provide all of the features and advantages set forth herein. Furthermore, various omissions, substitutions, and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed; others may be added. Accordingly, the scope of the present disclosure is not intended to be limited by the specific disclosures of preferred embodiments herein, and may be defined by claims as presented herein or as presented in the future. The language of the claims is to be interpreted broadly based on the language employed in the claims and not limited to the examples described in the patent specification of during prosecution of the application, which examples are to be construed as non-exclusive.

Features, materials, characteristics, or groups described in conjunction with a particular aspect, embodiment, or example are to be understood to be applicable to any other aspect, embodiment, or example described herein unless incompatible therewith. All of the features disclosed in this specification (including any accompanying claims, abstract, and drawings), or all of the steps of any method or process so disclosed, may be combined in any combination, except combinations where at least some of such features or steps are mutually exclusive. The protection extends to any novel one, or any novel combination, of the features disclosed in this specification (including any accompanying claims, abstract, and drawings), or to any novel one, or any novel combination, of the steps of any method or process so disclosed.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of the stated amount. As another example, the terms “generally parallel” and “substantially parallel” may refer to a value, amount, or characteristic that departs from exactly parallel by less than 14 degrees.

What is claimed is:

1. A rejuvenation system, the system comprising:
a percussive-impacting mechanism configured to generate an acceleration on the user;
an inspired-gas delivery unit configured to control a pressure and a composition of a gas inhaled by the user;
a vascular-constriction device configured to compress a blood vessel of the user; and
a nerve-plexus-stimulation device configured to generate a magnetic field adjacent a nerve plexus of the user.

2. The system of claim 1, wherein the percussive-impacting mechanism comprises a magnetic drive unit configured to pull a striker rod through a core of the magnetic drive unit.

3. The system of claim 1, wherein the percussive-impacting mechanism comprises a platform on which the patient stands to receive treatment from the percussive-impacting mechanism.

4. The system of claim 1, wherein the percussive-impacting mechanism comprises a chair on which the patient sits to receive treatment from the percussive-impacting mechanism.

5. The system of claim 1, wherein the percussive-impacting mechanism comprises a bed on which the patient lies to receive treatment from the percussive-impacting mechanism.

6. The system of claim 1, wherein the inspired-gas delivery unit comprises a nose cannula, or a face mask, or a hood sized to cover a head of the user.

7. The system of claim 1, wherein the vascular-constriction device comprises a cuff sized to wrap around an arm of the user.

8. A rejuvenation system comprising:
a percussive-impacting mechanism;
an inspired-gas delivery unit; and
a control unit electronically coupled with the percussive-impacting mechanism and the inspired-gas delivery unit, the control unit configured to control a magnitude or a frequency of a vibration generated by the percussive-impacting mechanism, the control unit further configured to control a partial pressure of a gas delivered by the inspired-gas delivery unit.

9. The system of claim 8, wherein the control unit is connected to a vascular-constriction device and further configured to control a duration or an intensity of a tissue compression delivered from the vascular-constriction device.

10. The system of claim 8, wherein the control unit is connected to a magnetic-field generator and further configured to control a strength of a magnetic field produced from the magnetic-field generator.

11. A method of treating a person with a rejuvenation system, the method comprising:
placing the person onto a percussive-impacting mechanism;
attaching a vascular-constriction device to the person;
generating a vibration with the percussive-impacting mechanism; and
compressing a tissue of the person with the vascular-constriction device, wherein generating the vibration and compressing the tissue are performed contemporaneously.

12. The method of claim 11, further comprising generating, with a nerve-plexus-stimulation device, a magnetic field near a nerve plexus of the person.

13. The method of claim 12, wherein the magnetic field has a strength between 5 mT and 1 T.

14. The method of claim 11, further comprising regulating, with an inspired-gas delivery unit, a partial pressure of a gas inhaled by the person.

15. The method of claim 14, wherein the gas is oxygen.

16. The method of claim 11, wherein a frequency of the vibration is between 10 Hz and Hz.

17. The method of claim 11, wherein a magnitude of the vibration is between 0.1 g and 10 g.

18. The method of claim 11, wherein the vascular-constriction device applies a tissue-compression pressure between 50 mmHg and 250 mmHg to the tissue of the person.

19. The method of claim 18, wherein the vascular-constriction device varies the tissue-compression pressure over time.

20. The method of claim 19, wherein a magnitude of the tissue-compression pressure varies over time in a sinusoidal fashion.
